SEQUENCE LISTING (26 sequences)

SEQ ID NO: 1 - DNA, 20 bp, Human papillomavirus type 18
gatggcaatccaataagtat

SEQ ID NO: 2 - DNA, 20 bp, Human papillomavirus type 39
gatgggtatgcaataagttt

SEQ ID NO: 3 - DNA, 20 bp, Human papillomavirus type 45
gatggtaatcctataagtat

SEQ ID NO: 4 - DNA, 20 bp, Human papillomavirus type 59
gatggcaatcctataagtgt

SEQ ID NO: 5 - DNA, 20 bp, Human papillomavirus type 68
gatggtaacccaataagttt

SEQ ID NO: 6 - DNA, 19 bp, Human papillomavirus type 16
gaaatgcattggatggaaa

SEQ ID NO: 7 - DNA, 19 bp, Human papillomavirus type 31
gaaatgcactagatggcaa

SEQ ID NO: 8 - DNA, 19 bp, Human papillomavirus type 33
gaaatgcgttagatggaaa

SEQ ID NO: 9 - DNA, 19 bp, Human papillomavirus type 35
gaaatgcactagatggaaa

SEQ ID NO: 10 - DNA, 19 bp, Human papillomavirus type 52
gaaatgcactggatggaaa

SEQ ID NO: 11 - DNA, 19 bp, Human papillomavirus type 58
gaaatgcattagatggtaa

SEQ ID NO: 12 - DNA, 22 bp, Human papillomavirus type 51
aatttttaagatatcaaggtgt

SEQ ID NO: 13 - DNA, 22 bp, Human papillomavirus type 56
aatttttaagatatcaaggggt

SEQ ID NO: 14 - DNA, 21 bp, Human papillomavirus type 18
cctttcaaaaaaacatttcca

SEQ ID NO: 15 - DNA, 21 bp, Human papillomavirus type 39
cttttcaaaaaaacatttcca

SEQ ID NO: 16 - DNA, 21 bp, Human papillomavirus type 45
cctttcaaaaaaacatttcca

SEQ ID NO: 17 - DNA, 21 bp, Human papillomavirus type 59
cctttcaaaaaaacatttcca

SEQ ID NO: 18 - DNA, 21 bp, Human papillomavirus type 68
cttttcaaaaaaacatttcca

SEQ ID NO: 19 - DNA, 19 bp, Human papillomavirus type 16
ggatttccgttttcgtcaa

SEQ ID NO: 20 - DNA, 19 bp, Human papillomavirus type 31
ggatttccgtttttgtcaa

SEQ ID NO: 21 - DNA, 19 bp, Human papillomavirus type 33
gggttaccattttcatcaa

SEQ ID NO: 22 - DNA, 19 bp, Human papillomavirus type 35
gggtttccatttttatcaa

SEQ ID NO: 23 - DNA, 19 bp, Human papillomavirus type 52
ggattgccattttcatcaa

SEQ ID NO: 24 - DNA, 19 bp, Human papillomavirus type 58
ggattaccatttgcatcaa

SEQ ID NO: 25 - DNA, 19 bp, Human papillomavirus type 51
ctagtggctgtaaccaaaa

SEQ ID NO: 26 - DNA, 19 bp, Human papillomavirus type 56
ctaatggctgcaaccaaaa

(19) United States
(12) Patent Application Publication
(10) Pub. No.: US 2010/0285472 A1
(43) Pub. Date: Nov. 11, 2010

(54) PRIMERS AND PROBES FOR DETECTION OF HIGH RISK GROUP GENO-TYPE HUMAN PAPILLOMAVIRUS DNA, A QUALITATIVE ASSAY METHOD OF THE SAME DNA USING THEM AND A QUALITATIVE ASSAY KIT OF THE SAME DNA

(76) Inventors: Hyunil Choi, Seoul (KR); Taewoo Kim, Wonju (KR)

Correspondence Address: MARTINE PENILLA & GENCARELLA, LLP, 710 LAKEWAY DRIVE, SUITE 200, SUNNYVALE, CA, 94085, US

(21) Appl. No.: 12/690,021

(22) Filed: Jan. 19, 2010

Related U.S. Application Data
(63) Continuation of application No. PCT/KR2007/003988, filed on Aug. 21, 2007.

(30) Foreign Application Priority Data
Jul. 19, 2007 (KR) 10-2007-0072306
Jul. 19, 2007 (KR) 10-2007-0072307

(51) Int. Cl. C12Q 1/68; C07H 21/00

(52) U.S. Cl. 435/6; 536/24.33; 536/24.32

(57) ABSTRACT

The present invention relates to a method for qualitative assay of high risk group Human Papillomavirus (HPV) DNA by PCR method using primers designed to detect the infection of 13 high risk group HPV which are closely associated with cervical cancer in a simple and precise way, and a kit for qualitative assay of high risk group HPV DNA.

### CLAIM OF PRIORITY

This application claims priority under 35 U.S.C. §§120 and 365(c) as a continuation application of prior International Application PCT/KR2007/003988, which was filed on Aug. 21, 2007, and which was published in English under PCT Article 21(2). The disclosure of the prior international application is incorporated herein by reference.

### TECHNICAL FIELD

The present invention relates to primers and probes for detecting DNA of 13 high risk group Human Papillomavirus (HPV) types, i.e., HPV-16, -18, -31, -33, -35, -39, -45, -51, -52, -56, -58, -59 and -68, a method for qualitative assay of said high risk group HPV DNA by PCR-hybridization using said primers and probes, and a qualitative assay kit of said high risk group HPV DNA.

### BACKGROUND ART

Human Papillomavirus (HPV) is a 8 kb double-stranded closed circular DNA virus. HPV consists of a viral particle (virion) in the shape of an icosahedron and approximately 8000 base pairs. HPV has a circular double stranded DNA molecule, said DNA molecule being surrounded by a capsid protein.

All types of HPV have open reading frames (ORFs), a portion of DNA that encodes proteins with similar properties. They are divided into early genes (E1, E2, E4, E5, E6 and E7) of approximately 4.5 kb and late genes (L1 and L2) of approximately 2.4 kb. The early genes have roles in viral DNA replication (E1), induction or suppression of the action of DNA encoding a protein which causes malignant transformation of host cells (E2), synthesis of a protein responsible for the growth of host cells and viruses (E4), induction of the activity of epidermal growth factor (EGF) and colony stimulator factor (CSF) receptors (E5), malignant transformation through permanent survival of cells, activation of oncogenes and inactivation of cancer suppressor genes (E7), etc. Particularly, it has been found that E6 and E7, oncogenic proteins which are expressed after HPV infects the epithelial cell of a host, bind to tumor suppressor proteins of host cells, p53 and pRB, respectively, thereby suppressing the functions of said tumor suppressor proteins, leading to the transformation of infected cells, thereby making the cells develop into tumors.

Meanwhile, the late genes comprise DNAs coding for viral major capsid proteins (L1) and viral minor capsid proteins (L2); and a long control region (LCR) of 1 kb, a non-coding region controlling the transcription and translation of the late genes.

HPV infects the cervix, vagina, vulva, urethra or the anal area of a woman, and more than about 70 types of HPV have been identified so far. HPV is divided into a high risk group and a low risk group according to its correlation with cancer and a mechanism where the tumor of cervical epithelium develops into a high risk group (CIN 2-3 (2˜3 stage cervical lesion)).

HPV-16 and -18 have been considered as a high risk group which causes cancer, and HPV-31, -33, -35 have proved to act as a mediator of cancer since they have been more often found in CIN 2-3 than in cancer. The cancers associated with HPV-16 or -18 occur more often than the cancers associated with HPV-31, -33, or -35, and about 80% of cervical cancer result from said five types of HPV. Beside the above HPV types, HPV-39, -45, -51, -52, -56, -58, -59, -68 also mediate cancer, falling within a high risk group.

HPV may remain latent in an adult for several years, and simultaneous infection of several types of HPV is possible, which is a major risk factor of cervical cancer.

Meanwhile, most of the HPV infection involves a reversible cell change. HPV DNA is found in about 10% of women having normal cervical epithelial cells, but actually, HPV DNA prevails in women within a group of certain ages and demographics. According to the results of a study conducted on women of ages of 16˜60, within two years after infection, 15˜28% of HPV positive women develop 2˜3 stage CIN or squamous intraepithelial lesion (SIL) which is considered to be a cancer, whereas only 1˜3% of HPV negative women develop such symptoms. Particularly, it has been confirmed that HPV-16 and -18 develop about 40% times faster than the other HPV types.

It is considered that high risk group HPV is a major factor causing a malignant tumor or cancer. In addition, it is considered that even if a woman is Pap and HR HPV negative, if the woman is persistently infected with one or more types of HPV or reinfected, the woman may become HPV positive or develop a precancerous cervical lesion. Methods for the early diagnosis of cervical cancer have been studied focusing on the correlation between HPV infection and cervical cancer.

Methods for the diagnosis of cervical cancer associated with HPV are classified into a cytological method and a molecular biological method.

A cytological method, which detects changes in the form of a HPV infected cell, has advantages in terms of economy, easiness in handling, safety, etc. However, a cytological method has low objectivity since it makes diagnosis based on observations of the form of cells. In addition, a cytological method does not produce persistent data due to its high rate of false-negative results (15˜45%).

Meanwhile, a molecular biological method is classified into a method of assaying HPV DNA directly and a method of assaying HPV DNA after amplification of the same by PCR. A molecular biological method has an advantage of being capable of objectively detecting virus DNA which is a cause of cervical cancer, thereby performing precise diagnosis. Accordingly, a molecular biological method has an advantage that, in case where the diagnosis by Papanicolaou (Pap) Smear test or the diagnosis by biopsy is unreliable, it can diagnose cervical cancer at an early stage prior to pathological diagnosis of cervical cancer.

In order to directly identify HPV DNA, methods using HPV type-specific probes, such as a southern blotting method, dot blotting method and a filter in situ hybridization (FISH), have been conventionally used. Recently, Hybrid Capture System (by Digene Corporation) has been developed and widely used. As compared with previous HPV DNA diagnosis method, Hybrid Capture System has high preciseness and has an advantage of diagnosing both high and low risk groups. However, because Hybrid Capture System requires large amount of HPV DNA, it has a limitation in detection in the case of diagnosing early stage HPV infection or in the case where an appropriate amount of sample has not been obtained. In addition, because Hybrid Capture System uses a RNA probe, it has low safety, and uses relatively expensive equipment, and has low analytical sensitivity.

A PCR method is a technology of detecting the presence of a pathogen by amplifying a tiny amount of the pathogen's nucleic acid using a complementary oligonucleotide (primer) which specifically binds to the base sequence of nucleic acid of the target pathogen (See U.S. Pat. Nos. 4,683,195 and 4,683,202). As compared with said cytological method and a method for direct assay of HPV DNA, a PCR method is considered to be superior in terms of preciseness, easiness and costs.

As for the primers used in a method for detecting HPV by PCR, general primers which can amplify HPV DNA regardless of the type of HPV and HPV-specific primers which can selectively amplify target HPV DNA have been developed so far.

However, in the case of using a general primer, even if an amplified product is obtained by PCR, it cannot be detected whether it is a high risk HPV type closely associated with cervical cancer. Thus, in the case of using a general primer, it is difficult to check the development of cervical cancer precisely. In other words, in the case of using a general primer which can complementarily bind to all types of HPV DNA, the general primer amplifies all types of HPV DNA, and in such case, it is difficult to check precisely how much the HPV infection has progressed or to anticipate how much area the cervical cancer has spread to. Thus, in the case of using a general primer, there is a limitation that a method such as the above-described Hybrid Capture System should be used to deal with such problems.

Meanwhile, in the case of using a HPV type-specific primer capable of specifically binding to each HPV type, a user suffer the inconvenience of having to use different primers in order to detect the presence of a certain HPV type in a sample. That is, in case where a patient is infected with a HPV type which cannot complementarily bind to a HPV type-specific primer, an amplified product of the HPV will not be obtained by PCR, and in such case, the patient may be diagnosed to be healthy notwithstanding that he is actually HPV infected. Thus, in the case of using a HPV type-specific primer, various primers have to be used simultaneously. Thus, a HPV-specific primer has a disadvantage that it cannot be used in a wide range of clinical studies.

Accordingly, consensus has been made that in order to increase practical efficiency of the diagnosis of cervical cancer based on HPV DNA amplification by PCR, a primer which selectively amplifies each of all of the high risk HPV types, which are known to have a high chance of developing into cervical cancer, and thereby can detect the infection of high risk group HPV at an early stage and diagnose cervical cancers resulting from high risk group HPV in a simple way at once needs to be developed.

Accordingly, in order to specifically detect HPV-16, -18, -31, -33, -35, -39, -45, -51, -52, -56, -58, -59, -68, which are classified into high risk group HPV types, the inventors conducted a research to select a gene specific to said genotypes of viruses, and as the result of the research, the inventors set E1 gene as such a gene. Based on this, the inventors found a primer which can diagnose the infection of said 13 high risk group HPV in a simple and sensitive way, and thereby completed the present invention.

### SUMMARY

The present invention suggests a way to solve said problems of conventional methods. An object of the present invention is to provide a primer and probe which can diagnose the infection of said 13 high risk group HPV which are closely associated with cervical cancer in a simple and sensitive way in the detection of HPV DNA by PCR.

Another object of the present invention is to provide a method of detecting the presence of said 13 high risk group HPV by amplifying target DNA using said primer and probe and checking whether the DNA has been amplified.

A further object of the present invention is to provide a qualitative test kit characterized by comprising said primers and probes.

### BRIEF DESCRIPTION OF DRAWINGS

FIG. 1 schematically shows a colorimetric method (using an enzyme immunoassay apparatus).

FIG. 2 schematically shows a luminescence method (using a chemiluminescence measurement apparatus).

### DETAILED DESCRIPTION

In order to achieve said objects, the present invention provides a primer capable of amplifying DNA of high risk group HPV type-16, -18, -31, -33, -35, -39, -45, -51, -52, -56, -58, -59, -68, consisting of:

an oligonucleotide having Sequence ID No. 1 and Sequence ID No. 14;
an oligonucleotide having Sequence ID No. 2 and Sequence ID No. 15;
an oligonucleotide having Sequence ID No. 3 and Sequence ID No. 16;
an oligonucleotide having Sequence ID No. 4 and Sequence ID No. 17;
an oligonucleotide having Sequence ID No. 5 and Sequence ID No. 18;
an oligonucleotide having Sequence ID No. 6 and Sequence ID No. 19;
an oligonucleotide having Sequence ID No. 7 and Sequence ID No. 20;
an oligonucleotides having Sequence ID No. 8 and Sequence ID No. 21;
an oligonucleotide having Sequence ID No. 9 and Sequence ID No. 22;
an oligonucleotide having Sequence ID No. 10 and Sequence ID No. 23;
an oligonucleotide having Sequence ID No. 11 and Sequence ID No. 24;
an oligonucleotides having Sequence ID No. 12 and Sequence ID No. 25;
an oligonucleotide having Sequence ID No. 13 and Sequence ID No. 26; and combinations thereof.

In addition, the present invention provides a probe for detecting high risk group HPV DNA, consisting of at least one oligonucleotides selected from the oligonucleotides having Sequence ID No. 14 to Sequence ID No. 26.

In addition, the present invention provides a method for qualitative assay of high risk group HPV DNA by using PCR hybridization technique, wherein said method can detect the presence of 13 high risk group HPV DNA in a simple and precise way by using said primers and probes. Said method has an advantage that it is applicable to both a chemiluminescent assay and an enzyme immunoassay.

In addition, the present invention relates to a kit for qualitative assay of high risk group HPV DNA, comprising said primers and probes.

Hereinafter, we will describe the present invention in more detail.

The primers used in the present invention have the base sequences present in E1 genes of high risk group HPV (HPV-16, -18, -31, -33, -35, -39, -45, -51, -52, -56, -58, -59, -68), and have the following conserved sequences.

| | | |
|---|---|---|
| | | HPV |
| Primer sequence | Primer location | type |

Sense primers

| | | |
|---|---|---|
| Biotin 5′-gat ggb waw | E1 gene/2555~2574 | HPV-18 |
| scw ata agt dt-3′ | E1 gene/2539~2558 | HPV-39 |
| | E1 gene/2513~2532 | HPV-45 |
| | E1 gene/2474~2493 | HPV-59 |
| | E1 gene/2413~2432 | HPV-68 |
| Biotin 5′-gaa atg cry trg | E1 gene/2473~2491 | HPV-16 |
| atg gwa a-3′ | E1 gene/2411~2429 | HPV-31 |
| | E1 gene/2467~2485 | HPV-33 |
| | E1 gene/2415~2433 | HPV-35 |
| | E1 gene/2461~2479 | HPV-52 |
| | E1 gene/2471~2489 | HPV-58 |
| Biotin 5′-aat ttt taa gat | E1 gene/2162~2183 | HPV-51 |
| atc aag gkg t-3′ | E1 gene/2185~2206 | HPV-56 |

Anti-sense primers

| | | |
|---|---|---|
| 5′-cyt ttc aaa aaa aca | E1 gene/2779~2759 | HPV-18 |
| ttt cca-3′ | E1 gene/2763~2743 | HPV-39 |
| | E1 gene/2737~2717 | HPV-45 |
| | E1 gene/2698~2678 | HPV-59 |
| | E1 gene/2637~2617 | HPV-68 |
| 5′-ggr ttd ccr ttt kya | E1 gene/2662~2644 | HPV-16 |
| tca a-3′ | E1 gene/2610~2582 | HPV-31 |
| | E1 gene/2656~2638 | HPV-33 |
| | E1 gene/2603~2585 | HPV-35 |
| | E1 gene/2650~2632 | HPV-52 |
| | E1 gene/2660~2642 | HPV-58 |
| 5′-cta rtg gct gya acc | E1 gene/2371~2353 | HPV-51 |
| aaa a-3′ | E1 gene/2394~2376 | HPV-56 |

(In the above tables, r represents g or a; y represents t or c; k represents g or t; s represents g or c; w represents g or t; b represents g, c, or t; and d represents a, g, or t.)

The sense primer of sequence No. 1 according to the present invention is based on positions 2555-2574 of the genome sequence of HPV-18; the anti-sense primer of sequence No. 14 is based on positions 2779-2759 of the genome sequence of HPV-18; the sense primer of sequence No. 2 is based on positions 2539-2558 of the genome sequence of HPV-39; the anti-sense primer of sequence No. 15 is based on positions 2763-2743 of the genome sequence of HPV-39; the sense primer of sequence No. 3 is based on positions 2513-2532 of the genome sequence of HPV-45; the anti-sense primer of sequence No. 16 is based on positions 2737-2717 of the genome sequence of HPV-45; the sense primer of sequence No. 4 is based on positions 2474-2493 of the genome sequence of HPV-59; the anti-sense primer of sequence No. 17 is based on positions 2698-2678 of the genome sequence of HPV-59; the sense primer of sequence No. 5 is based on positions 2413-2432 of the genome sequence of HPV-68; the anti-sense primer of sequence No. 18 is based on positions 2637-2617 of the genome sequence of HPV-68; the sense primer of sequence No. 6 is based on positions 2473-2491 of the genome sequence of HPV-16; the anti-sense primer of sequence No. 19 is based on positions 2662-2644 of the genome sequence of HPV-16; the sense primer of sequence No. 7 is based on positions 2411-2429 of the genome sequence of HPV-31; the anti-sense primer of sequence No. 20 is based on positions 2610-2582 of the genome sequence of HPV-31; the sense primer of sequence No. 8 is based on positions 2467-2485 of the genome sequence of HPV-33; the anti-sense primer of sequence No. 21 is based on positions 2656-2638 of the genome sequence of HPV-33; the sense primer of sequence No. 9 is based on positions 2415-2433 of the genome sequence of HPV-35; the anti-sense primer of sequence No. 22 is based on positions 2603-2585 of the genome sequence of HPV-35; the sense primer of sequence No. 10 is based on positions 2461-2479 of the genome sequence of HPV-52; the anti-sense primer of sequence No. 23 is based on positions 2650-2632 of the genome sequence of HPV-52; the sense primer of sequence No. 11 is based on positions 2471-2489 of the genome sequence of HPV-58; the anti-sense primer of sequence No. 24 is based on positions 2660-2642 of the genome sequence of HPV-58; the sense primer of sequence No. 12 is based on positions 2162-2183 of the genome sequence of HPV-51; the anti-sense primer of sequence No. 25 is based on positions 2371-2353 of the genome sequence of HPV-51; the sense primer of sequence No. 13 is based on positions 2185-2206 of the genome sequence of HPV-56; and the anti-sense primer of sequence No. 26 is based on positions 2394-2376 of the genome sequence of HPV-56. The probes or primers of the present invention may be synthesized by an automatic synthesis method, etc.

The probes used in the present invention are oligonucleotides which can complementarily bind to PCR products obtained using said primers, and said probes may be oligonucleotides of appropriate length present in the region between said sense primers and anti-sense primers in said genome sequences of HPV. Preferably, oligonucleotides having the sequence of the anti-sense primers of said sequence Nos. 14˜26 are used.

The probes or primers used in the present invention may be further modified to include a detectable label for diagnosis and probe. Variable types of labels for diagnosis and probe are well-known in the art. Appropriate labels for the present invention include biotin, DIG, etc, but are not restricted to such examples. By using such labels, a person skilled in the art could easily obtain labeled, modified probes or primers of the present invention.

In the case of using a mixture of pairs of said sense primers and anti-sense primers, a qualitative assay of said 13 high risk group HPV DNA can be done in a simple way at once.

According to the present invention, said 13 high risk group HPV DNA's are amplified using primers designed to be specific to the DNA's well-conserved sequences, and then the amplified products are hybridized with probes designed to be specific to them in a microplate well, and then the obtained products bind to antibody labeled by enzyme, and then the presence of said 13 high risk group HPV DNA is detected by measuring the intensity of color (light absorption) or luminescence (light emission) generated by the activity of the conjugated enzyme. Here, the 5′ end of a forward primer is attached with biotin, and accordingly, the amplified product is attached to a microwell by the binding of biotin-streptavidin. Meanwhile, a probe where DIG is attached at the 5′ end is complementary to the base sequence of the amplified strand of a forward primer. As a measurement method, any one of an enzyme immunoassay conducted after binding POD enzyme to DIG of the hybridized probe and an chemiluminescent assay conducted after binding AP enzyme to DIG of the hybridized probe may be used.

Thus, the chemical reagent kit used for the method for qualitative assay of high risk group HPV DNA according to the present invention is an excellent kit which can be used for a colorimetric method (using an enzyme immunoassay apparatus) or a luminescence method (using a chemiluminescence measurement apparatus) as needed. Said qualitative assay of HPV DNA is an assay which is necessary to observe a HPV-infected patient or to confirm the treatment effects of an anti-virus agent.

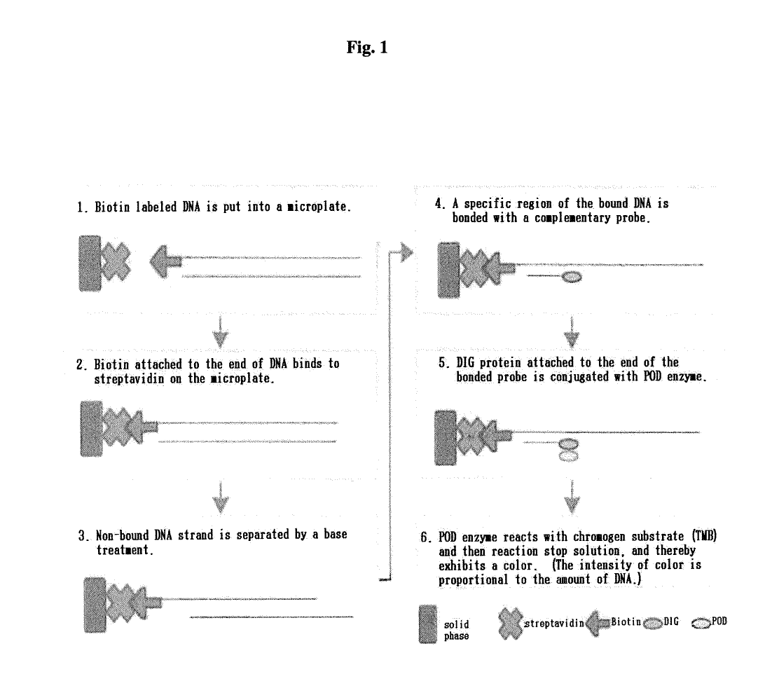

FIG. 1 schematically shows a colorimetric method (using an enzyme immunoassay apparatus). First, the DNA product of a specific base sequence is put into a microplate well, and then double-stranded DNA is attached to the surface of the microplate well by the bonding of biotin and streptavidin. Then, the DNA strand without biotin is separated, and then the attached DNA strand is hybridized with a probe labeled with DIG. The DIG binds to POD (peroxidase), and then the POD reacts with chromogen substrate TMB (tetra methyl benzidine) to exhibit a color. The intensity of color is measured by ELISA reader. The intensity of light absorption is proportional to the amount of HPV DNA.

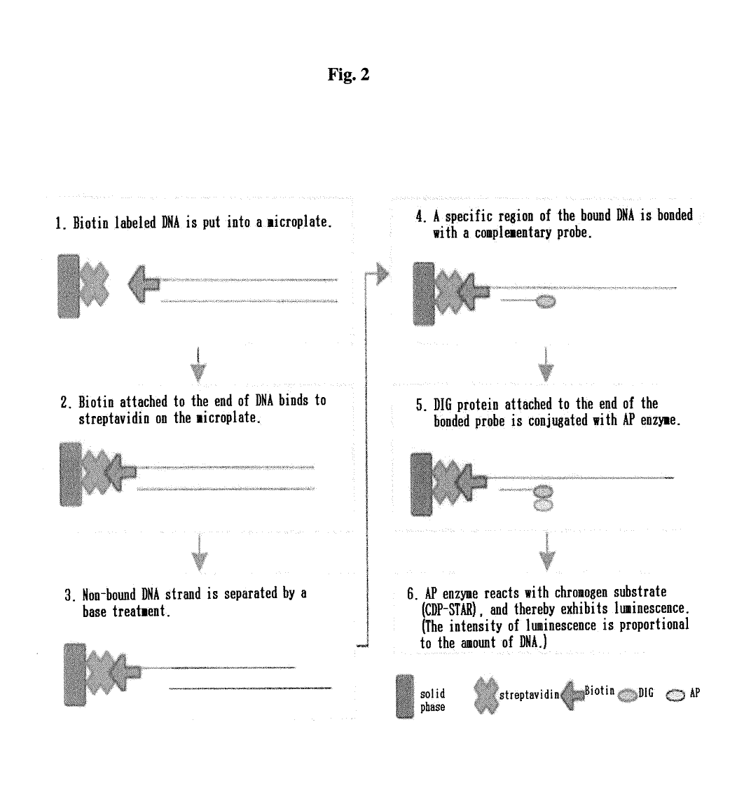

FIG. 2 schematically shows a luminescence method (using a chemiluminescence measurement apparatus). First, the DNA product of a specific base sequence is put into a microplate well, and then double-stranded DNA is attached to the surface of the microplate well by the bonding of biotin and streptavidin. Then, the DNA strand without Biotin is separated, and then the attached DNA strand is hybridized with a probe labeled with DIG. The DIG binds to AP (alkaline phosphatase), and then the AP reacts with chromogen substrate CDP-STAR to emit luminescence. The intensity of luminescence is measured by a luminometer. The intensity of luminescence is proportional to the amount of HPV DNA.

The following table shows the differences between a colorimetric method and a luminescence method. The processes of the two methods are identical except for said differences.

| | Colorimetric method | Luminescence method |
|---|---|---|
| Enzyme being used | Peroxidase (POD) | Alkaline phosphatase |
| | | (AP) |
| Substrate being used | Peroxide | CDP-star ™ |
| Equipment being used | ELISA reader | Luminometer |
| Standard reagent | 0, 5, 100, 1000, 2000 | 0, 5, 100, 2000, 5000 |
| concentration(pg/ml) | | |

### EFFECTS OF THE INVENTION

The present invention selects the sequences with high conservation probability from the base sequences present in E1 genes of 13 high risk group HPV (i.e., HPV-16, -18, -31, -33, -35, -39, -45, -51, -52, -56, -58, -59, -68) and designs primers based on them. By using the primers of the present invention, the infection of said 13 high risk group HPV can be detected in a simple and precise way. Particularly, by using a mixture of the primers of sequence Nos. 1˜26 of the present invention, the infection of said 13 high risk group HPV can be detected in a simple and precise way.

In addition, the kit for qualitative assay of high risk group HPV of the present invention allows to conduct an assay at a relatively low price in a simple way and to obtain more precise qualitative assay results as compared with Hybrid Capture 2 Test by Digene Corporation.

In addition, according to the present invention, costs resulting from the use of additional equipment etc. may be reduced since the measurement method of the present invention may be selected from any one of an enzyme immunoassay, conducted after binding POD enzyme to DIG of the hybridized probe, and an chemiluminescent assay, conducted after binding AP enzyme to DIG of the hybridized probe.

### Example

### Best Modes for Carrying Out the Invention

Now, the present invention will be described in further detail with reference to Examples. However, it should be understood that the examples set forth below are for the purpose of illustration and that the scope of the invention is not in any way limited to such specific Examples.

### I. Detection of High Risk Group HPV by Using Colorimetry Analysis

The presence of 13 high risk group HPV DNA in the specimen sampled from cervix can be detected by using an enzyme-linked immunosorbent assay. A sample is interpreted as positive if more than 1 pg/ml (100,000 copies/ml) of HPV DNA is present in the sample.

### 1. Reagent (Qualitative Assay Kit) for the Detection of High Risk Group HPV DNA According to the Present Invention

The reagent (qualitative assay kit) for detection of high risk group HPV DNA according to the present invention comprises the following features. However, it should be understood that the features of the reagent of the present invention are not restricted to the following features and that modifications may be made based on common knowledge in the art.

Kit for Extracting HPV DNA

| | |
|---|---|
| Lysis solution | 12 ml |
| Neutralizing solution | 12 ml |
| Microplate for extracting DNA (Clear) | 96 wells |

Biotin-Labeled DNA and a Kit for Hybridization

Standard Substance

| | |
|---|---|
| Negative control group | 0.4 ml |
| High risk group HPV positive calibrator | 0.4 ml each |

Biotin Labeling

| | |
|---|---|
| Reaction tube for labeling biotin | 96 T |
| (Tube used before amplification and | |
| mixing) | |
| Buffer for biotin labeling (amplification | 6 ml |
| buffer) | |
| Mixture of primers* | 0.5 ml |

*A mixture of oligonucleotides of sequence Nos. 1~26 of the present invention is used as a mixture of primers.

Hybridization Solution

| | |
|---|---|
| Diluent solution of biotin-labeled DNA | 16 ml |
| product (Diluent solution) | |
| Biotin-labeled DNA product binding | 16 ml |
| solution (Product binding solution) | |
| Denaturation solution | 12 ml |
| Hybridization buffer containing probes* | 18 ml |
| Microplate for hybridization (Clear) | 8 wells × 12 strips |
| Antibody conjugated enzyme (DIG | 12 ml |
| conjugated POD) | |
| Chromogen substrate (TMB) | 9 ml |
| Reaction stop solution (Stop sol.) | 9 ml |
| 10 × concentrated washing solution (wash | 50 ml |
| buffer) | |

*Oligonucleotides of sequence Nos. 14~26 where DIG is attached at the 5′end are used as probes.

### 2. Method for Qualitatively Assay of High Risk Group HPV DNA According to the Present Invention

Detection of high risk group HPV DNA using the primers of sequence Nos. 1˜26 of the present invention may be done according to the following steps. However, it should be understood that the detection method is not restricted to have the following steps and that modifications may be made based on common knowledge in the art.

### (1) Cervix Biopsy

Cervix biopsy was done such that the cross-section of the sample could be 2-5 mm thick. 1.0 ml of SDB (Sample Dilution Buffer) was immediately added to the specimen obtained by biopsy, and the thus-obtained mixture was stored at −20° C. For your reference, a specimen obtained by biopsy can be stored for at most 2 weeks at the room temperature, for 2˜3 weeks at 2-8° C., and for 3 weeks ˜3 months at −20° C.

### (2) Extraction of HPV DNA

500 μl of lysis reagent was added to a tube where a specimen is contained. The mixture was mixed by using a pipet, and then was incubated in a water bath at 37° C. for 1 hour. Then, 150 μl of neutralizing solution was put into each (clear) microplate for extracting DNA, and then 100 μl of said incubated reagent was added.

### (3) Amplification of Biotin-Labeled DNA Fragments

### 1) Preparation of a Biotin Labeling Solution

3 μl of primer was mixed with 37 μl of amplification buffer to obtain a biotin labeling solution. 40 μl of the thus-prepared biotin labeling solution was added to said mixture in a dry state, and then 10 μl of said extracted DNA was added to obtain a total of 50 μl of mixture.

### 2) Biotin Labeling Conditions

5 minutes at 95° C.
(30 seconds at 95° C., 30 seconds at 52° C., and 50 seconds at 72° C.)×40 cycles
5 minutes at 72° C.
stored at 4° C.

### (4) Hybridization

150 μl of diluent solution was added to 50 μl of biotin-labeled products and the mixture was mixed by using a pipet. 150 μl of product binding buffer was put into a well of a (clear) microplate for hybridization, and then 30 μl of said diluent biotin-labeled product was added and then mixed. Then, the well was tightly sealed, and the mixture was reacted in a water bath at 37° C. for 1 hour. After the reaction was completed, moisture was removed, and then sealing tapes were carefully removed. Then, 100 μl of denaturation solution was added to each well, and the wells were sealed, and then the mixture was reacted at the room temperature for 30 minutes. Meanwhile, a mixture of probes was prepared before the completion of denaturation reaction. After the completion of denaturation reaction, the product was washed with washing solution 3 times. After washing, the moisture in a microplate was removed by using filter paper. Then, 150 μl of hybridization buffer containing probes were injected into a microwell, and then mixed. Then, the well was sealed and the mixture was reacted at a water bath at 37° C. for 1 hour. After completion of reaction, the product was washed with washing solution 5 times. After washing, the moisture in a microplate was removed by using filter paper. Then, 100 μl of a solution of enzyme conjugated with antibody (DIG Conjugated POD) was put into the well and then the mixture was mixed. Then, the mixture was reacted in a water bath at 37° C. for 30 minutes. After completion of reaction, the product was washed with washing solution 5 times. After washing, the moisture in a microplate was removed by using filter paper. Then, 100 μl of chromogen substrate (TMB) was put into each well, and the mixture was reacted in a darkroom for 30 minutes. Then, 100 μl of reaction stop solution was injected into each well to stop color reaction. After the color reaction stopped, the intensity of color was measured by using ELISA reader.

Wavelength Used to Measure Color Development

Measurement wavelength: 450 nm, Standard wavelength: 620 nm

### 3. Results of the Qualitative Assay

A qualitative assay was conducted according to said method, using the kit for diagnosing high risk group HPV characterized in comprising primers of sequence Nos. 1˜26 of the present invention. Then, sensitivity, specificity, correlation between the reagent of the present invention and a conventional reagent (Hybrid Capture 2 Test by Digene Corporation), reproducibility, and whether there is a cross-reaction was assayed.

### (1) Assay of Sensitivity

HPV High Positive Control of Accromatrix Co. was purchased, and 184 positive specimens were prepared from them. Then, a qualitative assay was conducted by using the kit of the present invention. 92 specimens were assayed in an experiment, and such an experiment was repeated once more to assay a total of 184 specimens. After the assay of said 184 positive specimens, sensitivity (representing the ratio of positives) was calculated. Sensitivity of the kit of the present invention in this instance was 100%.

Sensitivity=number of true positives/(number of true positives+number of false negatives)×100(%)

### (2) Assay of Specificity

Using the kit of the present invention, a qualitative assay was conducted on 184 specimens which was negative by the test using the product of Digene Corporation sold in Korea. 92 specimens were assayed in an experiment, and such an experiment was repeated once more to assay a total of 184 specimens. After the assay of said 184 negative specimens, specificity (representing the ratio of negatives) was calculated. Of 184 specimens, 182 were found to be true negative, and only 2 were found to be false positive. That is, specificity of the kit of the present invention in this instance was 98.9%.

(3) Comparison with Type-Specific PCR

Type-specific PCR's capability of detecting high risk group HPV DNA from stored clinical specimen was measured. A total of 152 assays were conducted, and the results are shown in the following Table 1.

TABLE 1

| | | PCR | | |
|---|---|---|---|---|
| | | Positive | Negative | Total |
| Kit for qualitative | Positive | 65 | 11 | 76 |
| assay of HPV | Negative | 9 | 67 | 76 |
| according to the | Total | 74 | 78 | 152 |
| present invention | | | | |

(4) Correlation with a Conventional Reagent (Comparison with the Hybrid Capture 2 High-Risk Group Test by Digene Corporation)

In order to determine the correlation between the kit for qualitative assay of high risk group HPV of the present invention and a conventional reagent (Hybrid Capture 2 High-Risk Group Test by Digene Corporation), 620 times of experiments were conducted on the same specimens. Here, the specimens must include more than 40 abnormal specimens. The results of the experiments are shown in the following table 2. According to the comparison results of the kit for qualitative assay of HPV of the present invention with the product by Digene Corporation, the concordance rate was 85.32%.

TABLE 2

| | | Qualitative assay kit | | |
|---|---|---|---|---|
| | | of the present invention | | |
| | | Positive | Negative | Total |
| Hybrid Capture | Positive | 224 | 80 | 304 |
| 2 by Digene | Negative | 9 | 307 | 316 |
| Corporation | Total | 233 | 387 | 620 |

Sensitivity[positives/(positives+false negatives)]=73.68%(224/304)

Specificity[negatives/(negatives+false positives)]=97.15%(307/316)

Numerical value of positive results=96.13%(224/233)

Numerical value of negative results=79.32%(307/387)

Additional analysis was done on the 9 specimens which were false positive and 80 specimens which were false negative by the kit of Digene Corporation in said comparison experiment of the qualitative assay kit of the present invention. Said specimens were subjected to PCR using GP 5+/6+ and MY 09/11 primers (For detailed information of the primers, refer to publications on primers, etc.). Then, the PCR product was subjected to electrophoresis. Positive specimens showed bands at 150 by or 450 bp, and negative specimens did not show any band. The experimental results are shown in the following Table 3.

TABLE 3

| | | Correlation experiment results | | |
|---|---|---|---|---|
| | | False positive | False negative | Total |
| Electrophoresis | No band shown | 1 | 78 | 79 |
| results | 150 or 450 bp | 8 | 2 | 10 |
| | Total | 9 | 80 | 89 |

As can be seen from Table 3, of the 9 specimens which were positive by the kit for qualitative assay of the present invention but which were negative by Hybrid Capture 2 Test of Digene Corporation, 8 were found to be positive by PCR method using GP 5+/6+ and MY 09/11 primers. In addition, of the 80 specimens which were negative by the kit for qualitative assay of the present invention but which were positive by Hybrid Capture 2 Test of Digene Corporation, 78 were found to be negative by PCR method using GP 5+/6+ and MY 09/11 primers. That is, it was confirmed that the kit of the present invention have superior results in a qualitative assay over Hybrid Capture 2 Test Kit by Digene Corporation.

### (5) Assay of Reproducibility

In order to test the reproducibility of experiments with regard to a date, time of a day, and location and the overall reproducibility of the kit of the present invention using HPV positive and negative clinical specimens, reproducibility experiments were done at 2 different labs on 3 different days using the same amount of the kit of the present invention. The specimens used in the reproducibility experiments are as follows:

N1: Negative calibrator (0.2 pg/ml), loaded and assayed 15 times
N2: Negative calibrator (0.5 pg/ml), loaded and assayed 15 times
P3: low positive (1.0 pg/ml), loaded and assayed 15 times
P4: low positive (2.5 pg/ml), loaded and assayed 15 times
P5: medium positive (5.0 pg/ml), loaded and assayed 15 times
P6: high positive (10.0 pg/ml), loaded and assayed 15 times
The results of reproducibility experiments are shown in the following table.

According to Dates

| | Day 1 | | Day 2 | | Day 3 | | | |
|---|---|---|---|---|---|---|---|---|
| | Rate of | Rate of | Rate of | Rate of | Rate of | Rate of | Rate of | |
| | false | false | false | false | false | false | false | Rate of false |
| | negative | positive | negative | positive | negative | positive | negative | positive |
| | results | results | results | results | results | results | results | results |
| N1 | — | 0/15 | — | 0/15 | — | 0/15 | — | 0/45 |
| N2 | — | 0/15 | — | 1/15 | — | 0/15 | — | 1/45 |
| P3 | 0/15 | — | 0/15 | — | 0/15 | — | 0/45 | — |
| P4 | 1/15 | — | 0/15 | — | 0/15 | — | 1/45 | — |
| P5 | 0/15 | — | 0/15 | — | 1/15 | — | 1/45 | — |
| P6 | 0/15 | — | 0/15 | — | 0/15 | — | 0/45 | — |
| Total | 1/60 | 0/30 | 0/60 | 1/30 | 1/60 | 0/30 | 2/180 | 1/90 |

According to Locations

| | Lab 1 | | Lab 2 | | | |
|---|---|---|---|---|---|---|
| | Rate of | Rate of | Rate of | Rate of | Rate of | Rate of |
| | false | false | false | false | false | false |
| | negative | positive | negative | positive | negative | positive |
| | results | results | results | results | results | results |
| N1 | — | 0/15 | — | 0/15 | — | 0/30 |
| N2 | — | 0/15 | — | 0/15 | — | 0/30 |
| P3 | 0/15 | — | 0/15 | — | 0/30 | — |
| P4 | 0/15 | — | 1/15 | — | 1/30 | — |
| P5 | 0/15 | — | 0/15 | — | 0/30 | — |
| P6 | 0/15 | — | 0/15 | — | 0/30 | — |
| Total | 0/60 | 0/30 | 1/60 | 0/30 | 1/120 | 0/60 |

According to the Time of Day of Experiment

| | First | | | | | |
|---|---|---|---|---|---|---|
| | experiment | | Second experiment | | | |
| | Rate of | Rate of | Rate of | Rate of | Rate of | Rate of |
| | false | false | false | false | false | false |
| | negative | positive | negative | positive | negative | positive |
| | results | results | results | results | results | results |
| N1 | — | 0/15 | — | 0/15 | — | 0/30 |
| N2 | — | 0/15 | — | 0/15 | — | 0/30 |
| P3 | 0/15 | — | 0/15 | — | 0/30 | — |
| P4 | 0/15 | — | 0/15 | — | 0/30 | — |
| P5 | 0/15 | — | 0/15 | — | 0/30 | — |
| P6 | 0/15 | — | 0/15 | — | 0/30 | — |
| Total | 0/60 | 0/30 | 0/60 | 0/30 | 0/120 | 0/60 |

Overall Reproducibility

| | | Rate of | Rate of |
|---|---|---|---|
| | Number of | false | false |
| | the specimen | negative | positive |
| Specimen | concerned | results | results |
| N1 | 105 | — | 0/105 |
| N2 | 105 | — | 1/105 |
| All of the negative | 210 | — | 1/210 |
| specimens | | | |
| P3 | 105 | 0/105 | — |
| P4 | 105 | 2/105 | — |
| P5 | 105 | 1/105 | — |
| P6 | 105 | 0/105 | — |
| All of the positive | 420 | 3/420 | — |
| specimens | | | |

### (6) Cross-Reaction Experiment

4 ng/ml of hepatitis B virus, hepatitis C virus, viral DNA of HPV-1, -2, -3, -4 and -5, plasmid DNA or human serum were assayed using the kit of the present invention. All of them showed negative results.

### II. Detection of High Risk Group HPV by Using Chemiluminescence Analysis

The presence of 13 high risk group HPV DNA in the specimen sampled from cervix can be detected by using an chemiluminescence analysis. A sample is interpreted as positive if more than 1 pg/ml (100,000 copies/ml) of HPV DNA is present in the sample.

1. Reagent (Kit for Qualitative Assay) for Detection of High Risk Group HPV DNA According to the Present Invention which was Used in Chemiluminescence Analysis

The reagent (qualitative assay kit) for detection of high risk group HPV DNA according to the present invention which was used in chemiluminescence analysis is the same as the kit for qualitative assay of high risk group HPV DNA according to the present invention (Kit for extracting HPV DNA, Biotin-labeled DNA and Kit for hybridization) which was used in said experiment for detection of high risk group HPV by using colorimetry analysis, except that in the present analysis, DIG conjugated AP was used as an antibody-conjugated enzyme solution constituting a hybridization solution and CDP-STAR was used as a chromogen substrate and that reaction stop solution was not used.

### 2. Method for Qualitative Assay of High Risk Group HPV DNA According to the Present Invention

Detection of high risk group HPV DNA using primers of sequence Nos. 1˜26 of the present invention may be done according to the following steps. However, it should be understood that the detection method is not restricted to the following steps and that modifications may be made based on common knowledge in the art.

### (1) Cervix Biopsy

Cervix biopsy was done in the same way as that of said detection of HPV by using colorimetry analysis.

### (2) Extraction of HPV DNA

Extraction of HPV DNA was done in the same way as that of said detection of HPV by using colorimetry analysis.

### (3) Amplification of Biotin-Labeled DNA Fragments

Amplification of biotin-labeled DNA fragments was done in the same way as that of said detection of HPV by using colorimetry analysis.

### (4) Hybridization

150 μl of diluent solution was added to 50 μl of biotin-labeled products and the mixture was mixed by using a pipet. 150 μl of product binding buffer was put into a well of a (white) microplate for hybridization, and then 30 μl of said diluted biotin-labeled product was added and then mixed. Then, the well was tightly sealed, and the mixture was reacted in a water bath at 37° C. for 1 hour. After completion of the reaction, moisture was removed, and then sealing tapes were carefully removed. Then, 100 μl of denaturation solution was added to each well, and the wells were sealed, and then the mixture was reacted at the room temperature for 30 minutes. After completion of the reaction, the product was washed with washing solution 3 times. After washing, the moisture in a microplate was removed by using filter paper. Then, 150 μl of hybridization buffer containing probes were injected into a microwell, and then mixed. Then, the well was sealed and the mixture was reacted at a water bath at 37° C. for 1 hour. After completion of the reaction, the product was washed with washing solution 5 times. After washing, the moisture in a microplate was removed by using filter paper. Then, 100 μl of antibody-conjugated enzyme solution (DIG Conjugated AP) was put into the well and then the mixture was mixed. Then, the mixture was reacted in a water bath at 37° C. for 30 minutes. After completion of the reaction, the product was washed with washing solution 5 times. After washing, the moisture in a microplate was removed by using filter paper. Then, 100 μl of chromogen substrate (CDP-STAR) was put into each well, and the mixture was reacted in a darkroom for 15 minutes. Within 30 minutes after the stop of the luminescence reaction, the intensity of luminescence was measured by using luminometer.

### 3. Results of the Qualitative Assay

A qualitative assay was conducted according to said method, using the kit for diagnosing high risk group HPV characterized in comprising primers of sequence Nos. 1˜26 of the present invention. Then, sensitivity, specificity, correlation between the reagent of the present invention and a conventional reagent (Hybrid Capture 2 Test by Digene Corporation), reproducibility, and whether there is a cross-reaction was assayed.

### (1) Assay of Sensitivity

HPV High Positive Control of Accromatrix Co. was purchased, and 184 positive specimens were prepared from them. Then, a qualitative assay was conducted using the kit of the present invention. 92 specimens were assayed in an experiment, and the same experiment was repeated once more to assay a total of 184 specimens. After the assay of said 184 positive specimens, sensitivity (representing the ratio of positives) was calculated. Sensitivity of the kit of the present invention in this instance was 100%.

### (2) Assay of Specificity

Using the kit of the present invention, a qualitative assay was conducted on 184 specimens which were negative by the test using the product of Digene Corporation sold in Korea. 92 specimens were assayed in an experiment, and such an experiment was repeated once more to assay a total of 184 specimens. After the assay of said 184 negative specimens, specificity (representing the ratio of negatives) was calculated. Of 184 specimens, 182 were found to be true negative, and only 2 were found to be false positive. That is, specificity of the kit of the present invention in this instance was 98.9%.

### (3) Analytical Sensitivity

13 HPV DNA types (16, 18, 31, 33, 35, 39, 45, 51, 52, 56, 58, 59 and 68) were diluted to have HPV concentrations of 100 pg/ml, 10 pg/ml, 2.5 pg/ml, 1.0 pg/ml, 0.5 pg/ml and 0.2 pg/ml, and were used as specimens to be assayed. Then, analytical sensitivities of each of the HPV types were determined. The signal corresponding to the concentration of each of the HPV types was calculated in Relative Light Units (RLU). The data is as shown in the following Table 4.

TABLE 4

| | Minimum | | |
|---|---|---|---|
| | detectable | | |
| HPV | concentration | Standard | Coefficient |
| DNA | of HPV DNA | deviation | of variation |
| type | (pg/ml) | (SD) | (% CV) |
| 16 | 1.07 | 0.04 | 3.74 |
| 18 | 1.06 | 0.05 | 4.72 |
| 31 | 1.00 | 0.07 | 7.00 |
| 33 | 1.11 | 0.01 | 0.90 |
| 35 | 1.16 | 0.03 | 2.59 |
| 39 | 1.44 | 0.02 | 1.39 |
| 45 | 0.87 | 0.05 | 5.75 |
| 51 | 1.25 | 0.07 | 5.60 |
| 52 | 1.12 | 0.09 | 8.04 |
| 56 | 1.08 | 0.09 | 8.33 |
| 58 | 1.03 | 0.01 | 0.97 |
| 59 | 0.92 | 0.01 | 1.09 |
| 68 | 1.11 | 0.02 | 1.80 |
| Average | 1.09 | 0.04 | 3.94 |
| (All of | | | |
| the types) | | | |

(4) Comparison with Type-Specific PCR

Type-specific PCR's capability of detecting high risk group HPV DNA from a stored clinical specimen was measured. A total of 152 assays were conducted, and the results are as shown in the following Table 5.

TABLE 5

| | | PCR | | |
|---|---|---|---|---|
| | | Positive | Negative | Total |
| Kit for qualitative | Positive | 65 | 11 | 76 |
| assay of HPV | Negative | 9 | 67 | 76 |
| according to the | Total | 74 | 78 | 152 |
| present invention | | | | |

(5) Correlation with a Conventional Reagent (Comparison with a High Risk Group Test by Using Hybrid Capture 2 of Digene Corporation)

In order to determine the correlation between the kit for qualitative assay of high risk group HPV of the present invention and a conventional reagent (Hybrid Capture 2 High-Risk Group Test by Digene Corporation), 620 times of experiments were conducted on the same specimens. Here, the specimens must include more than 40 abnormal specimens. The results of the experiments are shown in the following table 6. According to the comparison results of the kit for qualitative assay of HPV of the present invention and the product by Digene Corporation, the concordance rate was 85.32%.

TABLE 6

| | | Kit for qualitative assay | | |
|---|---|---|---|---|
| | | of the present invention | | |
| | | Positive | Negative | Total |
| Hybrid Capture 2 | Positive | 224 | 80 | 304 |
| by Digene | Negative | 9 | 307 | 316 |
| Corporation | Total | 233 | 387 | 620 |

Sensitivity[positives/(positives+false negatives)]=73.68%(224/304)

Specificity[negatives/(negatives+false positives)]=97.15%(307/316)

Numerical value of positive results=96.13%(224/233)

Numerical value of negative results=79.32%(307/387)

Additional analysis was done on the 9 specimens which were false positive and 80 specimens which were false negative by the kit of Digene Corporation in said comparison experiment of the kit for qualitative assay of the present invention. Said specimens were subjected to PCR using GP 5+/6+ and MY 09/11 primers (For detailed information of the primers, refer to publications on primers, etc.). Then, the PCR product was subjected to electrophoresis. Positive specimens showed bands at 150 by or 450 bp, and negative specimens did not show any band. The results of the experiment are shown in the following Table 7.

TABLE 7

| | | Correlation experiment results | | |
|---|---|---|---|---|
| | | False positive | False negative | Total |
| Electrophoresis | No band shown | 1 | 78 | 79 |
| results | 150 or 450 bp | 8 | 2 | 10 |
| | Total | 9 | 80 | 89 |

As can be seen from Table 7, of the 9 specimens which were positive by the kit for qualitative assay of the present invention but which were negative by the test by using Hybrid Capture 2 of Digene Corporation, 8 were found to be positive by PCR method using GP 5+/6+ and MY 09/11 primers. In addition, of the 80 specimens which were negative by the kit for qualitative assay of the present invention but which were positive by Hybrid Capture 2 Test of Digene Corporation, 78 were found to be negative by PCR method using GP 5+/6+ and MY 09/11 primers. That is, it was confirmed that the kit of the present invention has superior results in a qualitative assay over Hybrid Capture 2 Test Kit by Digene Corporation.

### (6) Assay of Reproducibility

In order to test the reproducibility of experiments with regard to a date, time of a day, and location and the overall reproducibility of the kit of the present invention using HPV positive and negative clinical specimens, reproducibility experiments were done at 2 different labs on 3 different days using the same amounts of the kit of the present invention. The specimens used in the reproducibility experiments are as follows:

N1: Negative calibrator (0.2 pg/ml)
N2: Negative calibrator (0.5 pg/ml)
P3: low positive (1.0 pg/ml)
P4: low positive (2.5 pg/ml)
P5: medium positive (5.0 pg/ml)
P6: high positive (10.0 pg/ml)
The results of the reproducibility experiments are shown in the following table.

According to Dates

| | Day 1 | | Day 2 | | Day 3 | | RLU/CO | Rate of false |
|---|---|---|---|---|---|---|---|---|
| | Average | CV | Average | CV | Average | CV | of the | positive |
| | RLU/CO | (%) | RLU/CO | (%) | RLU/CO | (%) | total | results |
| N1 | 0.09 | 3.1 | 0.10 | 2.2 | 0.07 | 5.4 | 0.09 | 3.57 |
| N2 | 0.15 | 5.6 | 0.17 | 3.4 | 0.10 | 2.6 | 0.14 | 3.86 |
| P3 | 2.12 | 0.2 | 2.34 | 1.9 | 1.97 | 3.3 | 2.14 | 1.8 |
| P4 | 3.71 | 1.1 | 4.11 | 8.5 | 2.45 | 1.2 | 3.42 | 3.6 |
| P5 | 12.4 | 0.5 | 13.0 | 4.7 | 10.9 | 0.7 | 12.10 | 1.96 |
| P6 | 20.6 | 3.4 | 19.8 | 1.0 | 19.9 | 0.9 | 20.10 | 1.76 |
| Total | — | 2.31 | — | 3.61 | — | 2.35 | — | 2.75 |

According to Locations

| | | | | | Rate of | Rate of |
|---|---|---|---|---|---|---|
| | Lab 1 | | Lab 2 | | false | false |
| | Average | | Average | | negative | positive |
| | RLU/CO | CV (%) | RLU/CO | CV (%) | results | results |
| N1 | 0.09 | 3.57 | 0.08 | 4.1 | 0.09 | 3.84 |
| N2 | 0.14 | 3.86 | 0.15 | 2.4 | 0.15 | 3.13 |
| P3 | 2.14 | 1.8 | 2.33 | 0.5 | 2.24 | 1.15 |
| P4 | 3.42 | 3.6 | 4.41 | 3.31 | 3.92 | 3.46 |
| P5 | 12.10 | 1.96 | 13.50 | 1.74 | 12.8 | 1.85 |
| P6 | 20.10 | 1.76 | 19.94 | 5.41 | 20.02 | 3.59 |
| Total | — | 2.75 | — | 2.91 | — | 2.83 |

According to Time of Day of Experiment

Rate of

| | First | | | | Rate of | |
|---|---|---|---|---|---|---|
| | experiment | | Second experiment | | false | Rate of false |
| | Average | | Average | | negative | positive |
| | RLU/CO | CV (%) | RLU/CO | CV (%) | results | results |
| N1 | 0.08 | 3.18 | 0.10 | 3.02 | 0.09 | 3.1 |
| N2 | 0.16 | 9.7 | 0.14 | 1.5 | 0.15 | 5.6 |
| P3 | 2.41 | 0.2 | 1.83 | 0.2 | 2.12 | 0.2 |
| P4 | 4.45 | 1.19 | 2.97 | 1.01 | 3.71 | 1.1 |
| P5 | 13.56 | 0.75 | 11.24 | 0.25 | 12.4 | 0.5 |
| P6 | 21.90 | 2.48 | 19.30 | 4.32 | 20.6 | 3.4 |
| Total | — | 2.92 | — | 1.72 | — | 2.31 |

Overall Reproducibility

| | Number | | | | |
|---|---|---|---|---|---|
| | of the | | | | HPV |
| | specimen | Average | | HPV | negative |
| Specimen | concerned | RLU/CO | CV (%) | positive (%) | (%) |
| N1 | 36 | 0.09 | 3.84 | 0 (0%) | 36 (100%) |
| N2 | 36 | 0.14 | 3.13 | 0 (0%) | 36 (100%) |
| All of the | 72 | | | 0 (0%) | 72 (100%) |
| negative | | | | | |
| specimens | | | | | |
| P3 | 36 | 2.14 | 1.15 | 36 (100%) | 0 (0%) |
| P4 | 36 | 3.42 | 3.46 | 36 (100%) | 0 (0%) |
| P5 | 36 | 12.10 | 1.85 | 36 (100%) | 0 (0%) |
| P6 | 36 | 20.10 | 3.59 | 36 (100%) | 0 (0%) |
| All of the | 144 | | | 144 (100%) | 0 (0%) |
| positive | | | | | |
| specimens | | | | | |

### (7) Cross-Reaction Experiment

4 ng/ml of hepatitis B virus, hepatitis C virus, viral DNA of HPV-1, -2, -3, -4 and -5, plasmid DNA or human serum were assayed using the kit of the present invention. All of them showed negative results.

### Sequence List

Nucleotide of sequence ID No. 1 has the base sequence of positions 2555-2574 of the genome sequence of HPV-18.
Nucleotide of sequence ID No. 2 has the base sequence of positions 2539-2558 of the genome sequence of HPV-39.
Nucleotide of sequence ID No. 3 has the base sequence of positions 2513-2532 of the genome sequence of HPV-45.
Nucleotide of sequence ID No. 4 has the base sequence of positions 2474-2493 of the genome sequence of HPV-59.
Nucleotide of sequence ID No. 5 has the base sequence of positions 2413-2432 of the genome sequence of HPV-68.
Nucleotide of sequence ID No. 6 has the base sequence of positions 2473-2491 of the genome sequence of HPV-16.
Nucleotide of sequence ID No. 7 has the base sequence of positions 2411-2429 of the genome sequence of HPV-31.
Nucleotide of sequence ID No. 8 has the base sequence of positions 2467-2485 of the genome sequence of HPV-33.
Nucleotide of sequence ID No. 9 has the base sequence of positions 2415-2433 of the genome sequence of HPV-35.
Nucleotide of sequence ID No. 10 has the base sequence of positions 2461-2479 of the genome sequence of HPV-52.
Nucleotide of sequence ID No. 11 has the base sequence of positions 2471-2489 of the genome sequence of HPV-58.
Nucleotide of sequence ID No. 12 has the base sequence of positions 2162-2183 of the genome sequence of HPV-51.
Nucleotide of sequence ID No. 13 has the base sequence of positions 2185-2206 of the genome sequence of HPV-56.
Nucleotide of sequence ID No. 14 has the antisense sequence of positions 2779-2759 of the genome sequence of HPV-18.
Nucleotide of sequence ID No. 15 has the antisense sequence of positions 2763-2743 of the genome sequence of HPV-39.
Nucleotide of sequence ID No. 16 has the antisense sequence of positions 2737-2717 of the genome sequence of HPV-45.
Nucleotide of sequence ID No. 17 has the antisense sequence of positions 2698-2678 of the genome sequence of HPV-59.
Nucleotide of sequence ID No. 18 has the antisense sequence of positions 2637-2617 of the genome sequence of HPV-68.
Nucleotide of sequence ID No. 19 has the antisense sequence of positions 2662-2644 of the genome sequence of HPV-16.
Nucleotide of sequence ID No. 20 has the antisense sequence of positions 2610-2582 of the genome sequence of HPV-31.
Nucleotide of sequence ID No. 21 has the antisense sequence of positions 2656-2638 of the genome sequence of HPV-33.
Nucleotide of sequence ID No. 22 has the antisense sequence of positions 2603-2585 of the genome sequence of HPV-35.
Nucleotide of sequence ID No. 23 has the antisense sequence of positions 2650-2632 of the genome sequence of HPV-52.
Nucleotide of sequence ID No. 24 has the antisense sequence of positions 2660-2642 of the genome sequence of HPV-58.
Nucleotide of sequence ID No. 25 has the antisense sequence of positions 2371-2353 of the genome sequence of HPV-51.
Nucleotide of sequence ID No. 26 has the antisense sequence of positions 2394-2376 of the genome sequence of HPV-56.

1. Primers for detecting high risk group Human Papillomavirus (HPV) DNA selected from the following oligonucleotides:
an oligonucleotide having Sequence ID No. 1 and Sequence ID No. 14;
an oligonucleotide having Sequence ID No. 2 and Sequence ID No. 15;
an oligonucleotide having Sequence ID No. 3 and Sequence ID No. 16;
an oligonucleotide having Sequence ID No. 4 and Sequence ID No. 17;
an oligonucleotide having Sequence ID No. 5 and Sequence ID No. 18;
an oligonucleotide having Sequence ID No. 6 and Sequence ID No. 19;
an oligonucleotide having Sequence ID No. 7 and Sequence ID No. 20;
an oligonucleotide having Sequence ID No. 8 and Sequence ID No. 21;
an oligonucleotide having Sequence ID No. 9 and Sequence ID No. 22;
an oligonucleotide having Sequence ID No. 10 and Sequence ID No. 23;
an oligonucleotide having Sequence ID No. 11 and Sequence ID No. 24;
an oligonucleotide having Sequence ID No. 12 and Sequence ID No. 25;
an oligonucleotide having Sequence ID No. 13 and Sequence ID No. 26; and combinations thereof.

2. Primers for detecting high risk group HPV DNA according to claim 1, wherein the high risk group HPV DNA is DNA of HPV-type 16, type 18, type 31, type 33, type 35, type 39, type 45, type 51, type 52, type 56, type 58, type 59 or type 68.

3. Primers for detecting high risk group HPV DNA characterized by containing all of the oligonucleotides according to claim 1.

4. Primers for detecting high risk group HPV DNA according to claim 3, wherein the high risk group HPV DNA is DNA of HPV-type 16, type 18, type 31, type 33, type 35, type 39, type 45, type 51, type 52, type 56, type 58, type 59 or type 68.

5. Probes for detecting high risk group HPV DNA consisting of at least one oligonucleotides selected from the oligonucleotides of Sequence ID No. 14 to Sequence ID No. 26 according to claim 1.

6. Probes for detecting high risk group HPV DNA according to claim 5, wherein the high risk group HPV DNA is DNA of HPV-type 16, type 18, type 31, type 33, type 35, type 39, type 45, type 51, type 52, type 56, type 58, type 59 or type 68.

7. A method for qualitative assay of high risk group HPV DNA by using PCR hybridization technique, characterized by using primers according to claim 1.

8. The method for qualitative assay of high risk group HPV DNA according to claim 7, wherein the high risk group HPV DNA is DNA of HPV-type 16, type 18, type 31, type 33, type 35, type 39, type 45, type 51, type 52, type 56, type 58, type 59 or type 68.

9. A kit for qualitative assay of high risk group HPV DNA characterized by comprising primers according to claim 1.

10. The kit for qualitative assay of high risk group HPV DNA according to claim 9, wherein the high risk group HPV DNA is DNA of HPV-type 16, type 18, type 31, type 33, type 35, type 39, type 45, type 51, type 52, type 56, type 58, type 59 or type 68.

11. A method for qualitative assay of high risk group HPV DNA by using PCR hybridization technique, characterized by using probes according to claim 5.

12. A kit for qualitative assay of high risk group HPV DNA characterized by comprising probes according to claim 5.
